(19) United States
(12) Patent Application Publication
(10) Pub. No.: US 2010/0010395 A1
(43) Pub. Date: Jan. 14, 2010

(54) ULTRASOUND EMITTING SYSTEM AND ULTRASOUND TREATMENT MACHINE COMPRISING SAID SYSTEM

(76) Inventors: Cédric Gagnepain, Pringy (FR); Jean-Marc Andre, Saint Alban Leysse (FR)

Correspondence Address: KENYON & KENYON LLP, 1500 K STREET N.W., SUITE 700, WASHINGTON, DC, 20005, US

(21) Appl. No.: 12/443,035

(22) PCT Filed: Sep. 27, 2007
(86) PCT No.: PCT/FR07/52035

§ 371 Date: Mar. 26, 2009

(30) Foreign Application Priority Data
Sep. 27, 2006 (FR) 0653965

(51) Int. Cl. A61N 7/00

(52) U.S. Cl. 601/2

(57) ABSTRACT

An ultrasound emission system comprising a substantially cylindrical body and, inside said body: a piezoelectric assembly for producing ultrasound along the axial direction of said body, a sonotrode assembly, a prestress ring, and further comprising electrical power supply and control means; said piezoelectric assembly being constituted by a stack of layers of piezoelectric material, each layer being provided with excitation electrodes and presenting thickness lying in the range 20 μm to 100 μm. The system, generally fitted with a cannula enabling the ultrasound treatment to be applied to a determined area, can itself be incorporated in such a manner as to make an ultrasound treatment machine serving in particular to perform phacoemulsification.

This is a 371 national phase application of PCT/FR2007/052035 filed 27 Sep. 2007, claiming priority to French Patent Application No. 06/53965 filed 27 Sep. 2006, the contents of which are incorporated herein by reference.

### BACKGROUND OF THE INVENTION

The present invention relates to an ultrasound emission system. Such a system may be incorporated in an ultrasound or other treatment machine for treating a surface, by using ultrasound to destroy and remove fragile material, in particular certain kinds of biological tissue. One known application consists in incorporating such a system in a phacoemulsification machine. Such a machine can be used for performing a cataract operation. This operation consists in acting on the lens of an eye, once the lens has become opaque and therefore needs to be destroyed, so it can be replaced by an artificial lens that is transparent. The phacoemulsification machine enables the lens to be destroyed by ultrasound and enables the debris thereof to be removed in a single operation, thereby minimizing trauma for the eye and the patient.

In order to be able to perform its functions, the ultrasound emission system is made as follows: the system comprises a circuit enabling a stream of transparent fluid, generally an aqueous solution, to be directed to the surface for treatment. It also generates electrode seeking to destroy the materials that are to be removed. The ultrasound is conveyed by the fluid and strikes the surface for treatment. Materials that are fragile when subjected to ultrasound are then emulsified (destroyed and fragmented). The debris thereof detaches from the surface and becomes incorporated in the fluid. The fluid loaded with the debris is then sucked away and removed.

To make such an ultrasound emission system, it is known to make use of a substantially cylindrical body presenting a longitudinal axis, and presenting inside said body:
a piezoelectric assembly for producing vibration in said axial direction;
a sonotrode assembly or “sonotrode” for amplifying the vibration produced by said piezoelectric assembly, and mounted to move inside said body; and
a prestress ring, said piezoelectric assembly being mounted in the axial direction between said sonotrode and said prestress ring.

The system also includes electrical power supply and control means for applying an alternating voltage to said piezoelectric assembly.

In addition, at its front end, it includes a cannula that extends it and makes it possible to act on a surface in front of the cylindrical body.

Thus, with that system, piezoelectric materials are used not for imposing and maintaining constant movement with a large amount of force (e.g. deforming mirrors in the aerospace field), but for emitting ultrasound, which constitutes a function that is completely different.

In order to make a piezoelectric assembly, it is known for the piezoelectric assembly to make use of a ceramic that is said to be “massive” because it is constituted by a single layer.

In order to generate vibration by the piezoelectric effect with the help of such a massive ceramic, it is usually necessary to apply a power supply voltage that is high, i.e., of the order of 500 volts, root mean square (Vrms), or 1000 V peak to peak. Such a voltage is needed in particular to obtain movement with an amplitude close to 100 micrometers (μm) at the end of the above-mentioned cannula.

Because of the high electrical voltage, the ultrasound emission system is classified as at the boundary between low voltage and high voltage, using the terms employed by the (French) work code.

The applicable safety distances in air thus lie in the range 30 centimeters (cm) to 2 meters (m). Such a system thus presents a potential danger in the event of malfunction or degradation of the isolation, and it needs to be handled with care.

### SUMMARY OF THE INVENTION

The object of the invention is to remedy the above-mentioned drawback by enabling the power supply voltage of the ultrasound emission system to be reduced.

This object is achieved by the fact that said piezoelectric assembly is constituted by a stack of layers of piezoelectric material, each layer being provided with excitation electrodes and being of a thickness lying in the range 20 μm to 100 μm. By means of the piezoelectric effect, these various layers generate ultrasound and are thus referred to as emission layers.

The piezoelectric assembly is preferably powered over a frequency range close to a resonant frequency for the vibrating parts, i.e. the piezoelectric assembly, the sonotrode, and the hand-piece, which frequency thus also depends on the housing and on the cannula. Operating in such a frequency range enables the conversion of electrical power into mechanical power to be performed efficiently.

Advantageously, it has been found that in spite of the continuous vibratory stress to which the piezoelectric assembly is subjected during use, its layers of piezoelectric material turn out to be remarkably durable or solid in spite their small thickness (and generally the presence of a hole through the center thereof).

In addition, the vibratory behavior of the stack of layers is also found to be very satisfactory. The presence of a large number of excitation electrodes interposed between the layers of piezoelectric material, where such electrodes give rise to a corresponding number of mechanical interfaces between the layers, is not found to be penalizing in terms of producing the desired ultrasound wave at the end of the piezoelectric assembly.

The use of such a stack of piezoelectric layers makes it possible to limit the power supply voltage to an alternating voltage lying in the range 1 Vrms to 50 Vrms. Thus, the system presents low electrical risk and may be classified in the “very low voltage” category using the above-mentioned terminology.

Furthermore, the system frequently operates at high frequency, e.g. in the range 40 kilohertz (kHz) to 50 kHz. At such frequencies, the perception threshold of an electrical current, if any, is approximately 100 milliamps (mA), as compared with 10 mA at low frequency. That is why, the ultrasound emission system is safer than other systems operating at the same voltage but at low frequency, while nevertheless conserving its performance in terms of ultrasound generation.

Another point of this innovation relates to a piezoelectric detector element being integrated inside said body and being coupled to the piezoelectric assembly so as to deliver an electrical signal representative of the vibrations delivered thereby, e.g. representative of the amplitude and/or the periodicity of said vibration. Such a sensor is constituted merely by one or more layers of piezoelectric material similar to the other layers; however, instead of being excited and biased by the excitation electrodes and thus contributing to emitting ultrasounds waves by the piezoelectric effect, this layer is connected to a control portion of the power supply and control means; unlike the other layers, it acts as a sensor and delivers a signal that is a function of the vibration applied thereto.

The sensor made in this way acts in real time to evaluate the vibration generated within the system, e.g. to evaluate the amplitude and the periodicity of the vibration, with this being applicable regardless of the load or the action at the end of the system.

By modulating the excitation frequency of the piezoelectric assembly, it is possible, depending on the intended effect, either to increase the amplitude of the vibration by coming closer to the resonant frequency of the elements coupled in vibration within the system, or to reduce the amplitude by moving away from that frequency.

Naturally, the power supply voltage may also be modulated so as to take action also on the intensity of the ultrasound emission. These means for regulating the ultrasound emission by using said sensor provide effectiveness that is increased relative to traditional methods in which decisions are based on the voltage and the current fed to the piezoelectric assembly and also on the phase difference therebetween.

The invention can be well understood and its advantages appear better on reading the following detailed description of an embodiment given by way of non-limiting example.

### BRIEF DESCRIPTION OF THE DRAWINGS

The description refers to the accompanying drawings, in which:

FIG. 1 is a diagrammatic view of a phacoemulsification hand-piece incorporating an ultrasound emission system in accordance with the invention;

FIG. 2 is a section through the ultrasound emission system of the invention;

FIG. 3 is a simplified diagram of the power supply and control means of the ultrasound emission system of the invention; and

FIG. 4 shows a few layers of piezoelectric material so as to show how the piezoelectric assembly 1 is shaped, one of the layers being shown partially cut away.

### DETAILED DESCRIPTION

The description below of a preferred embodiment of the invention relates to a phacoemulsification hand-piece incorporating an ultrasound emission system of the invention. Nevertheless, such an ultrasound emission system can clearly be used for other applications and in other types of machine.

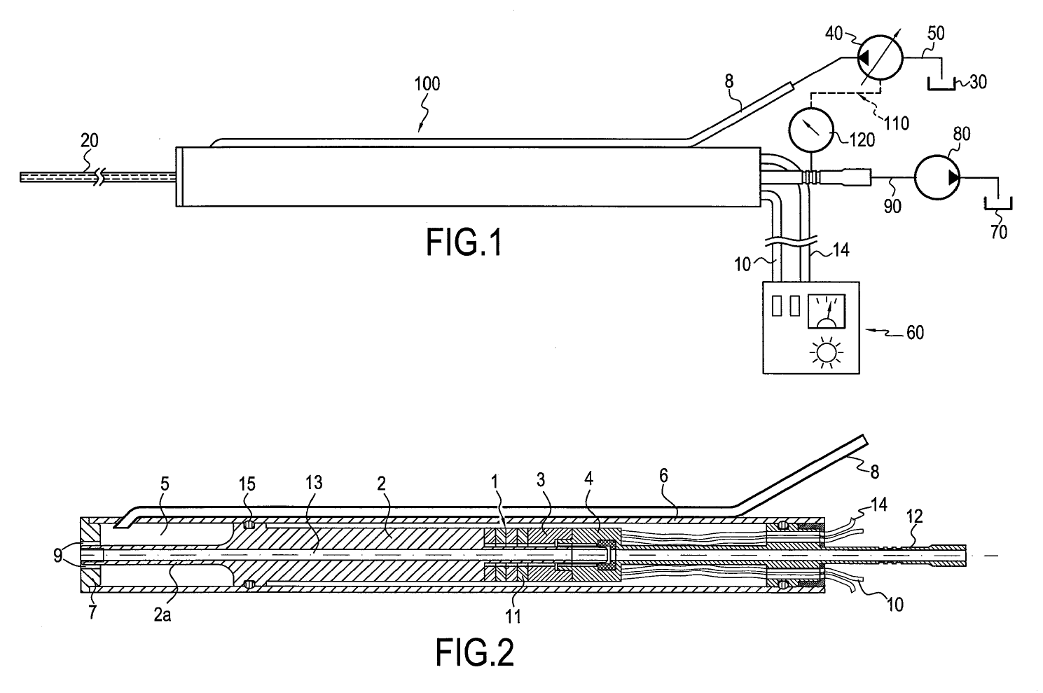

With reference to FIG. 1, there follows a description of an ultrasound treatment machine of the invention. It comprises a cannula 20, an ultrasound emission system 100; and for fluid feed: a tank 30, a pump 40, and a pipe 50; for fluid removal: a tank 70, a pump 80, and a pipe 90; electrical power supply and control means 60; and a device 110 for controlling the pump 40 and making use of a pressure gauge 120.

The cannula 20 is fastened to the front end of the ultrasound emission system 100. It comprises an outer cylindrical sheath for injecting fluid towards the surface that is to be cleaned, and an inner cylindrical needle for sucking up fluid from said surface.

The fluid is pumped from the tank 30 by the pump 40. On passing along the pipe 50, it is injected into the ultrasound emission system 100. On passing therethrough, it is delivered by the cannula 20 to the surface for cleaning. It becomes charged with debris generated by the ultrasound on said surface. It is then sucked up by the cannula 20 and returns into the ultrasound emission system 100. It is pumped therefrom by the pump 80 through the pipe 90 to the tank 70.

The pumps used may operate in various ways, for example it is possible to use a Venturi pump, an open-circuit peristaltic pump, a closed-circuit peristaltic pump, an eccentric pump, etc.

On the path of the fluid, its flow is regulated by the regulator device 110. The flow rate of the pump 40 is adjusted as a function of the flow rate of the pump 80 so as to guarantee that the zone for cleaning is supplied with sufficient fluid but not excessive fluid. For this purpose, the flow rate of the pump 40 is determined by the pressure gauge 120 located in the fluid removal pipe 90.

Electrically, the system 100 is connected to the power supply and control means by power supply cables 14 and control cables 10.

With reference to FIG. 2, there follows a more detailed description of the structure of the ultrasound emission system.

The main portion of the system is housed in a cylindrical body 6 and it comprises the following parts: a piezoelectric assembly 1; a sonotrode 2; a prestress ring 3; the rear suction part 4; and secondary parts.

The sonotrode 2 presents a central portion extending in the center of the ultrasound emission system, and rear and front portions that are substantially tubular and of diameters that are substantially smaller than that of the central portion and that extend from opposite sides thereof respectively towards the rear and towards the front of the system along its axis (the axis of the body 6).

Furthermore, the ultrasound emission system has a fluid feed pipe 8. It penetrates through an opening situated in the front portion of the body 6 so as to enable fluid to be delivered into a chamber 5 surrounding the tubular front portion of the sonotrode 2. At the front, the chamber 5 of the body 6 is closed by a plug 7. The plug include channels 9 that allow fluid to pass from the chamber 5 onto the outer sheath of the cannula 20. The plug 7 also includes an axial opening passing the cannula 20 and connecting it in leaktight manner to the sonotrode 2. Fluid return takes place via the inside of the cannula, in an inner needle contained in the outer sheath. Coming from the cannula, the fluid penetrates into the inner channel 13 that extends from one end to the other of the body 6 along its axis and that passes through the sonotrode 2 and the rear part 4. The fluid is sucked from there via the pipe 90 by the suction pump 80 and delivered to the tank 70.

The rear suction part 4 is secured to the cylindrical body 6. It is adhesively bonded to the rear end thereof which it closes. It comprises a cylindrical endpiece 12 extending rearwards onto which the fluid suction pipe 90 is connected. The rear suction part also has the above-mentioned inner channel 13 passing therethrough along its entire length.

The rear suction part is also the fastening point of the sonotrode 2. To enable such fastening, the rear tubular part of the sonotrode 2 has an outside thread and the rear suction part 4 has, towards its front, an opening with an inside thread. The rear portion of the sonotrode 2 is screwed into the rear suction part 4.

The prestress ring 3 and the piezoelectric assembly are fastened to the sonotrode and the rear suction part 4. They include cylindrical internal bores (or openings) corresponding to the outside shape of the rear tubular portion of the sonotrode. Thus, the prestress ring 3 (placed beside the rear suction part) and the piezoelectric assembly 1 can be threaded onto the rear tubular portion of the sonotrode; they are interposed between the central portion of the sonotrode 2 and the rear suction part 4 when the sonotrode is screwed on. Tightening the screw fastening of the sonotrode 2 enables the piezoelectric assembly 1 to be put into a state of light axial compression along the axis of the body 6, as is required to enable it to operate. The prestress ring 3 also acts as a washer and distributes the shear stresses generated by screw tightening. Furthermore, it is made of a material selected to optimize the operation of the piezoelectric assembly, making it possible in particular for the energy delivered by the piezoelectric assembly 1 to be transmitted towards the front of the system and not towards the rear.

The piezoelectric assembly 1, the sonotrode 2, the prestress ring 3, and the front portion of the rear suction part 4 are mounted to move in the body 6 so as to make it easier to emit ultrasound.

The cables 14 serve to power the piezoelectric assembly electrically. Under the effect of this power, the piezoelectric assembly 1 responds by the piezoelectric effect and generates ultrasound vibration. This vibration is communicated to the sonotrode 2 and propagates essentially towards the front of the system.

The sonotrode serves in particular to amplify the vibration. For this purpose, it presents a central portion presenting a large area of contact with the piezoelectric assembly so as to pick up as much as possible of the vibration that it emits. This central portion may be substantially cylindrical in shape and may have the same diameter as the piezoelectric assembly.

The sonotrode also has a junction portion that is substantially conical, connecting its central portion to its front tubular portion. The great reduction in diameter between the central portion and the front portion of the sonotrode has the advantageous consequence of strongly amplifying the amplitude of the ultrasound vibration that is transmitted towards the front of the cylindrical body and the cannula.

Furthermore, an O-ring gasket 15 surrounds the sonotrode inside the cylindrical body 6. It provides sealing, preventing the fluid from flowing from the chamber 5 around the central portion of the sonotrode and thus reaching the rear portion of the body 6 where the electric cables are to be found.

Finally, as mentioned above, a piezoelectric detector element 11 may be coupled to the piezoelectric assembly so as to perform a piezoelectric sensor function. This detector element may optionally have the same characteristics and the same electrodes as the other layers. It may comprise a plurality of layers of piezoelectric material, or it may be constituted by a single layer of piezoelectric material (of the “massive” type); it may be considerable thickness, e.g. up to more than one millimeter.

Advantageously, this piezoelectric detector element 11 is placed between the prestress ring 3 and the piezoelectric assembly 1.

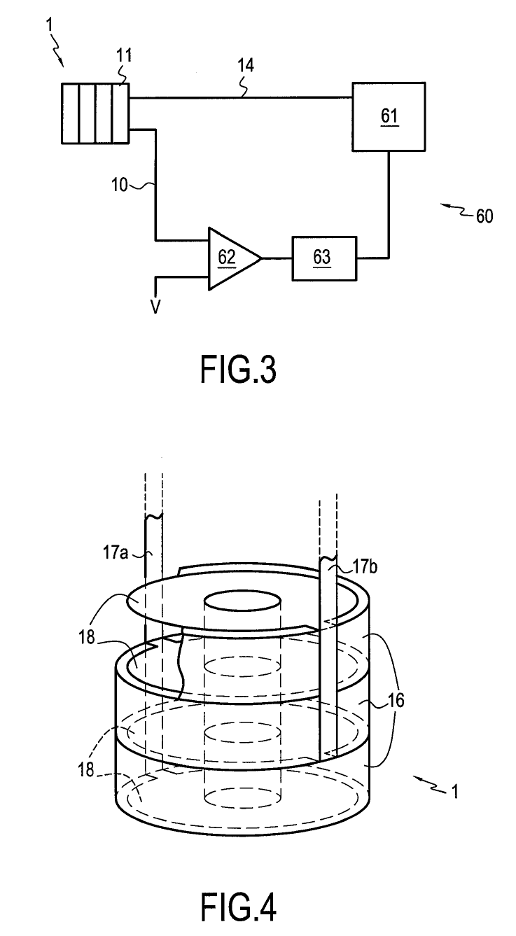

With reference to FIG. 3, there follows a description of the electrical power supply and control means of the system of the invention. Said means powers the piezoelectric assembly 1 via the cables 14; it receives information from the piezoelectric sensor via the cables 10. The cables 10 and the cables 14 extend inside the cylindrical body 6 to the electrodes, as shown in FIG. 2.

In order to enable the piezoelectric assembly to be regulated effectively, and thus in order to ensure that the ultrasound emission system operates effectively, the electrical power supply and control means 60 comprise:
electrical power supply means 61;
means 62 for comparing the signal delivered by the piezoelectric detector element 11 with reference values V; and
means 63 (a control circuit in the example shown) for determining the frequency and/or the voltage of the electrical signal to be applied to the piezoelectric assembly as a function of the results of said comparison and to transmit corresponding control setpoints to the above-mentioned power supply means 61.

With reference to FIG. 4, there follows a description of the structure of the piezoelectric assembly 1.

As mentioned above, this assembly is constituted by a stack of layers of piezoelectric material, each layer being provided with excitation electrodes 18. Advantageously, these layers are thin and may present thickness lying in the range 20 μm to 100 μm. For ease of understanding, only three layers of piezoelectric material 16 and their electrodes 17a, 17b, 18 are shown, and the top layer is shown partially cut away.

The layers may be made from various ceramics, and in particular from a sintered material based on lead titanozirconate (PZT).

Naturally, in the embodiment shown, the section of the body is circular and the layers of piezoelectric material are in the form of disks, each having a circular central opening. It will be understood that the section of the ultrasound emission system may be of arbitrary shape, this shape being reproduced by the layers of piezoelectric material.

The layers are powered electrically by two external electrodes 17a and 17b or edge electrodes, one positive and one negative. These edge electrodes serve to convey electricity from the cables 14 to the internal electrodes 18. In the cylinder constituted by the piezoelectric assembly, the edge electrodes generally occupy disjoint angular sectors so that they do not come into contact.

Each internal electrode 18 is substantially in the form of a disk that is thin relative to the thickness of the layers of piezoelectric material, and of diameter slightly smaller than the diameter of the layers 16. Each also includes a radial extension towards an edge electrode for connection thereto. Conversely, each internal electrode remains isolated from the other edge electrode, since it presents a diameter that is smaller than the diameter of the piezoelectric layers and therefore cannot come into contact with the edge electrode. The internal electrodes are placed between the layers of piezoelectric material and at the ends of the stack of layers of piezoelectric material. They are connected in alternation to the positive edge electrode and to the negative edge electrode.

Thus, each layer of piezoelectric material is biased by the two internal electrodes at opposite potentials on either side thereof, thereby contributing to generating vibration by the piezoelectric effect, at the rate and as a function of the electrical oscillations transmitted by the electrodes.

The above-described embodiment relates to an ultrasound treatment machine for cleaning biological tissue, such as for example a phacoemulsification system for use in ophthalmic surgery. Nevertheless, it should be understood that the system of the invention can be used in any ultrasound treatment machine, in particular for all types of cleaning operation whether on biological tissue or other tissue, the tissue to be removed possibly being adipose tissue or fat, calculi, etc.

1. An ultrasound emission system comprising:
a substantially cylindrical body presenting a longitudinal axis, and inside said body:
a piezoelectric assembly for producing vibration along said axial direction;
a sonotrode assembly for amplifying the vibration produced by said piezoelectric assembly, and mounted to move in said body; and
a prestress ring,
said piezoelectric assembly being mounted axially between said sonotrode assembly and said prestress ring; and
an electrical power supply and control device for applying an alternating voltage to said piezoelectric assembly;
wherein said piezoelectric assembly is constituted by a stack of layers of piezoelectric material, each layer being provided with excitation electrodes and presenting thickness lying in the range 20 μm to 100 μm.

2. A system according to claim 1, wherein said electrical power supply and control device delivers an alternating voltage lying in the range of 1 Vrms to 50 Vrms.

3. A system according to claim 1 further comprises further comprising a piezoelectric detector element inside said body and coupled to said piezoelectric assembly to deliver an electrical signal representative of the vibration delivered thereby.

4. A system according to claim 1, further comprising a piezoelectric detector element inside said body and coupled to said piezoelectric assembly to deliver an electrical signal representative of the amplitude and/or of the periodicity of the vibration delivered thereby.

5. A system according to claim 3 wherein said piezoelectric detector element is mounted between said piezoelectric assembly and said prestress ring.

6. A system according to claim 1, wherein said piezoelectric detector element presents thickness in the axial direction of at least 1 mm.

7. A system according to claim 3, wherein said piezoelectric detector element comprises a single layer of piezoelectric material.

8. A system according to claim 3, wherein said piezoelectric detector element comprises a plurality of layers of piezoelectric material.

9. A system according to claim 3, wherein the electrical power supply and control device comprises:
an electrical power supply device;
a device that compares the signal delivered by the piezoelectric detector element with reference values; and
a device that determines the frequency and/or the voltage of the electrical signal for applying to the piezoelectric assembly as a function of the results of said comparison, and that transmits corresponding control setpoints to the above-mentioned power supply device.

10. A system according to claim 1, wherein said piezoelectric assembly contains sintered material based on lead titanozirconate.

11. An ultrasound treatment machine comprising a system according to claim 1; a cannula mounted thereon; and a tank, a pump, and a pipe for supplying fluid; a tank, a pump, and a pipe for removing fluid; and a device for regulating the pump.

12. The application of the ultrasound treatment machine according to claim 11 for making a phacoemulsification assembly.
